(19) United States
(12) Patent Application Publication
(10) Pub. No.: US 2025/0342222 A1
(43) Pub. Date: Nov. 6, 2025

(54) PARAMETER GENERATION DEVICE, SYSTEM, METHOD, AND PROGRAM

(71) Applicant: NEC Corporation, Minato-ku, Tokyo (JP)

(72) Inventors: Akihiro YATABE, Tokyo (JP); Hiroshi CHISHIMA, Tokyo (JP); Masaru INABA, Tokyo (JP); Akio TODA, Tokyo (JP)

(73) Assignee: NEC Corporation, Minato-ku, Tokyo (JP)

(21) Appl. No.: 18/855,393

(22) PCT Filed: Apr. 28, 2022
(86) PCT No.: PCT/JP2022/019388

§ 371 Date: Oct. 9, 2024

(51) Int. Cl. G06F 17/11; G06Q 10/04; G06Q 50/04

(52) U.S. Cl. CPC G06F 17/11; G06Q 10/04; G06Q 50/04

(57) ABSTRACT

The input means 81 accepts input of a first objective function and a constraint condition defining a combination of factors related to item production. The objective function generation means 82 generates a second objective function by applying stochastic fluctuation to a parameter of the first objective function. The optimization processing means 83 performs optimization of a model including the second objective function and the constraint condition. The output means 84 outputs a value of a variable of the second objective function obtained by the optimization as a parameter set.

### TECHNICAL FIELD

The present invention relates to a parameter generation device, a parameter generation system, a parameter generation method, and a parameter generation program for generating desired a parameter.

### BACKGROUND ART

In research site for new item exploration, huge combinations of factors such as item type, amount, processing temperature, pressure, and time are tried to discover production methods that yield desired performance in new materials. However, the number of these combinations is astronomically large, making it impossible to try all of them.

Generally, from past experiences and simulation results, knowledge has been accumulated on combinations of conditions that can be expected to produce good results and vice versa. Therefore, in research site, the work of determining new combinations of factors within the range that satisfies the conditions indicated by this accumulated knowledge, and verifying the results through prototyping, simulations, etc., is repeated.

In addition, in order to derive a combination of factors that satisfies desired conditions, a mathematical programming solver may be used to derive an optimal combination based on an objective function designed by an engineer or the like and constraint conditions that define the conditions to be satisfied (i.e., a mathematical optimization problem).

Patent Literature 1 describes a design support system that reduces the number of times numerical simulations are performed in examining design parameters for achieving a design target. The design support system described in Patent Literature 1 performs sensitivity analysis to the design target by performing forward analysis given the initial set values of design parameters and then performing inverse analysis based on the results of the analysis based on the accompanying numerical analysis.

### CITATION LIST

### Patent Literature

Patent Literature 1: Domestic re-publication of PCT international application WO2007/122677

### SUMMARY OF INVENTION

### Technical Problem

In considering a method for producing the desired new item, it is necessary to create a combination of factors for the production of the new item. One such method is to rely on the experience and intuition of skilled personnel. However, this method is highly dependent on individuals, and there is a problem of being unable to carry out work efficiently without specific skilled personnel.

As another method for creating a combination of factors, a method of selecting a combination that satisfies a condition from among combinations of factors selected randomly can be considered. However, as the conditions become more complex, the probability of a combination of factors that satisfies the conditions decreases, resulting in poor efficiency.

On the other hand, it is possible to derive an optimal solution for a designed mathematical optimization problem by using a mathematical programming solver. However, the optimal solution obtained is one for the designed objective function. Usually, in situations where new items are being explored, some combination of types is needed as a candidate for the factors to product the item. Therefore, it is also inefficient for engineers to design a mathematical optimization problem each time a combination of factors is derived.

Additionally, the method described in Patent Literature 1 aims to reduce the number of simulations when considering design parameters to achieve design target and does not derive multiple combinations of factors.

Furthermore, there are cases where the objective function designed by engineers, etc. may not be entirely accurate. In such cases, there is also the problem that the obtained optimal solution may not necessarily be the solution for producing the desired item. Therefore, it is desirable to be able to efficiently discover multiple methods for producing desired items.

The purpose of this invention is to provide a parameter generation device, a parameter generation system, a parameter generation method, and a parameter generation program that can discover multiple methods for producing the desired item.

### Solution to Problem

The parameter generation device according to the present invention includes an input means which accepts input of a first objective function and a constraint condition defining a combination of factors related to item production, an objective function generation means which generates a second objective function by applying stochastic fluctuation to a parameter of the first objective function, an optimization processing means which performs optimization of a model including the second objective function and the constraint condition, and an output means which outputs a value of a variable of the second objective function obtained by the optimization as a parameter set.

The parameter generation system according to the present invention includes a predictive model generation device which uses past experimental data as training data to learn a predictive model with material as explanatory variable and characteristic value indicating a property of an item as objective variable, a first objective function generation device which generates a first objective function defining a combination of factors related to item production using the predictive model, and a parameter generation device which generates a parameter set using the first objective function, wherein the first objective function generation device generates the first objective function, including a linear sum of the characteristic value indicated by the objective variable as a combination of factors, and inputs it into the parameter generation device, and the parameter generation device includes: an input means which accepts input of the first objective function and a constraint condition, an objective function generation means which generates a second objective function by applying stochastic fluctuation to a parameter of the first objective function, an optimization processing means which performs optimization of a model including the second objective function and the constraint condition, and an output means which outputs a value of a variable of the second objective function obtained by the optimization as a parameter set.

The parameter generation method by a computer according to the present invention includes: accepting input of a first objective function and a constraint condition defining a combination of factors related to item production: generating a second objective function by applying stochastic fluctuation to a parameter of the first objective function; performing optimization of a model including the second objective function and the constraint condition; and outputting a value of a variable of the second objective function obtained by the optimization as a parameter set.

The parameter generation program according to the present invention causes a computer to execute: an input process for accepting input of a first objective function and a constraint condition defining a combination of factors related to item production, an objective function generation process for generating a second objective function by applying stochastic fluctuation to a parameter of the first objective function, an optimization processing process for performing optimization of a model including the second objective function and the constraint condition, and an output process for outputting a value of a variable of the second objective function obtained by the optimization as a parameter set.

### Advantageous Effects of Invention

According to the present invention, it becomes possible to discover multiple methods for producing the desired item.

### BRIEF DESCRIPTION OF DRAWINGS

FIG. 1 It depicts a block diagram showing an example of the configuration of an example embodiment of a simulation system of the present invention.

FIG. 2 It depicts an explanatory diagram showing an example of the operation of the parameter generation device.

FIG. 3 It depicts a block diagram showing an outline of the parameter generation device according to the present invention.

FIG. 4 It depicts a block diagram showing an outline of the simulation system according to the present invention.

FIG. 5 It depicts a schematic block diagram showing the configuration of a computer according to at least one example embodiment.

### DESCRIPTION OF EMBODIMENTS

Hereinafter, example embodiments of the present invention will be described with reference to the drawings.

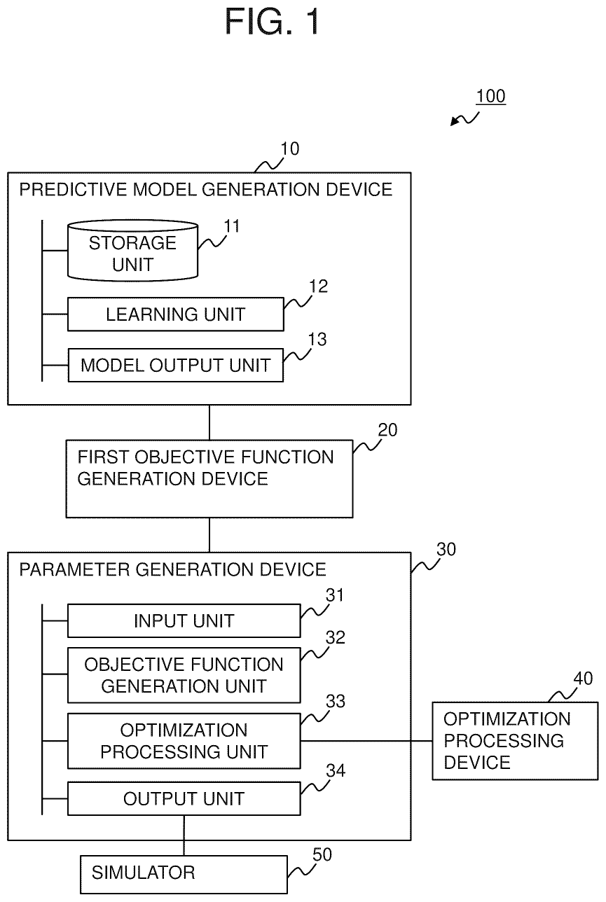

FIG. 1 is a block diagram showing an example of the configuration of an example embodiment of a simulation system of the present invention. The simulation system 100 of the present example embodiment includes a predictive model generation device 10, a first objective function generation device 20, a parameter generation device 30, an optimization processing device 40, and a simulator 50.

The predictive model generation device 10 is a device that generates a predictive model that predicts the impact of the type and amount of materials on the characteristics of a product (e.g., items) based on past experimental data. Specifically, the predictive model generation device 10 learns a predictive model that predicts value indicating item characteristics (hereinafter referred to as “characteristic value”) based on past experimental data. The characteristic value may also be referred to as performance indicator.

The predictive model generation device 10 includes a storage unit 11, a learning unit 12, and a model output unit 13.

The storage unit 11 stores training data used by the learning unit 12 for learning. The training data, for example, is data that corresponds to multiple materials used in the production of an item and property values that indicate the item's characteristics such as hardness, toughness, and heat resistance when those materials are used. The storage unit 11 may be realized, for example, by a magnetic disk, etc.

The learning unit 12 learns a predictive model with material as explanatory variable and characteristic value as objective variable using past experimental data as training data. The method by which the learning unit 12 learns the predictive model is arbitrary and may use any method, such as machine learning.

The model output unit 13 outputs the predictive model generated by the learning unit 12. The model output unit 13 may input the predictive model into the first objective function generation device 20.

The first objective function generation device 20 generates an objective function that defines a combination of factors related to item production to achieve target characteristic values. Here, the factors related to the item production mean the contents to be specified in the production method of the item, and specifically, they mean the type and amount of materials, processing temperature, pressure, processing time, etc. The objective function is defined using parameters such as weights (coefficients) and biases set for each factor.

In other words, the first objective function generation device 20 generates an objective function (hereinafter referred to as the first objective function) used to derive the optimal combination of material type, amount, processing method, etc. that achieves the target value, using the factors related to the production of the item and the parameters described above.

The first objective function generation device 20 may generate the first objective function using the predictive model generated by the learning unit 12. For example, it is assumed that the predictive model for predicting the i-th characteristic value yᵢ is expressed as a linear sum of j factors xⱼ, as shown in Equation 1 below; as a combination of factors. Here, x-bar (x with an overline) is the average value of the factors, and σ is the standard deviation of the factors, which are calculated when generating the predictive model.

$$\begin{matrix}[ Math . 1 ] & \\ {y}_{i} = {\sum }_{j} {a}_{ij} ( \frac{{x}_{j} - {\overline{x}}_{j}}{{σ}_{j}} ) + {b}_{i} & ( Equation 1 )\end{matrix}$$

In this case, the first objective function generation device 20 may generate the first objective function that includes the linear sum of each characteristic value yᵢ. For example, when the weight for each characteristic value yᵢ is Wᵢ, the first objective function generation device 20 may generate the first objective function as shown in Equation 2 below. Equation 2 represents the squared linear sum of the deviations from the target median value of the characteristic values. Here, Lmedᵢ is the target median value of the characteristic value yᵢ. Additionally, Weight Wᵢ is determined by engineers, etc. The specification of Wᵢ may be accepted by the first objective function generation device 20 or by the parameter generation device 30 described later.

$$\begin{matrix}[ Math . 2 ] & \\ {H}_{o} = {\sum }_{i} {( \frac{{y}_{i} - {Lmed}_{i}}{{Lmed}_{i}} )}^{2} \cdot {W}_{i} & ( Equation 2 )\end{matrix}$$

Furthermore, as shown in Equation 3 below, the first objective function may be expressed in the form of an expansion of Equation 2 above.

$$\begin{matrix}[ Math . 3 ] & \\ {H}_{o} = {\sum }_{i} {\sum }_{j \geq i} {Q}_{ij} {x}_{i} {x}_{j} + {\sum }_{i} {L}_{i} {x}_{i} + const . & ( Equation 3 )\end{matrix}$$

In the examples from Equations 1 to 3 above, aᵢⱼ, bᵢ, Lmedᵢ, Wᵢ, Qᵢⱼ, and Lᵢ are the parameters described above. In other words, the parameters in this example embodiment include not only the parameters at the time of formulation, but also the parameters obtained during the formulation process.

In the above explanation, the first objective function is configured as a squared linear sum of the deviations from the target median value of the objective variables (characteristic values), but the content included in the first objective function is not limited to characteristic values. The first objective function may include factors other than characteristic values (e.g., processing methods). The first objective function generation device 20 inputs the generated first objective function into the parameter generation device 30.

In this example embodiment, a case in which the first objective function generation device 20 is realized as an independent device is illustrated. However, the first objective function generation device 20 may be realized integrally with another device, and may be included in the parameter generation device 30, for example.

The parameter generation device 30 is a device that generates a parameter to be input into the simulator 50 and is connected to the optimization processing device 40 and the simulator 50. The simulator 50 is a device that performs trials based on the generated parameters. The aspect of the simulator 50 is arbitrary and may be implemented using known devices.

The optimization processing device 40 is a device that performs optimization processing based on the model generated by the parameter generation device 30. The optimization processing device 40 may be realized by a (classical) computer that executes a mathematical programming solver. Alternatively: the optimization processing device 40 may be a dedicated device that finds the ground state of the Hamiltonian of an Ising model. In this case, the optimization processing device 40 is realized as a device that executes annealing based on the Ising model generated by the parameter generation device 30.

The parameter generation device 30 includes an input unit 31, an objective function generation unit 32, an optimization processing unit 33, and an output unit 34.

The input unit 31 accepts input of the first objective function mentioned above. The input unit 31 also accepts input of constraint conditions indicating constraints that each factor must satisfy and constraints when combining factors. The input unit 31 may accept the first objective function generated by the first objective function generation device 20, or it may accept the first objective function generated manually by other devices (not shown) or engineers, etc.

For example, constraint conditions for producing new items include specifications regarding the selection of material types (one from each material group, etc.), specifications regarding the distribution of material quantities (specifying the sum of the quantities of some materials, specifying the quantities of individual materials, etc.), and specifications regarding exclusive materials. Other constraint conditions on the production of new items may include specifications for processing the material (depending on the material, there are restrictions on the processing temperature (upper limit temperature, etc.) and pressure, etc.).

The objective function generation unit 32 generates an objective function (hereinafter referred to as the second objective function) by applying stochastic fluctuations to the parameters of the input first objective function. Here, applying fluctuations to the parameters means performing calculation processing such as addition, subtraction, multiplication, or division on the values indicated by the fluctuations in parameters. The objects for which fluctuations are set include parameters that appear in the final first objective function (e.g., Qᵢⱼ, Lᵢ in the above Equation 3) as well as parameters that are in the process of being formulated (e.g., aᵢⱼ, Lmedᵢ in the above Equation 1).

The parameters to which fluctuations are applied are specified in advance. The specification method is arbitrary, and, for example, the input unit 31 may accept input from engineers, etc., for specifying the parameters to which fluctuations are applied. The fluctuations are applied to the parameters of the objective function, not the constraint conditions.

Specifically, the objective function generation unit 32 applies fluctuations represented by random variables following a predetermined probability distribution to the parameters of the first objective function. Preferably, the objective function generation unit 32 applies fluctuations represented by random variables following a probability distribution with mean zero to the parameters of the first objective function. Examples of probability distributions with mean zero include a normal distribution as exemplified by the following Equation 4 and a uniform distribution as exemplified by Equation 5.

$$\begin{matrix}[ Math . 4 ] & \\ p ( x ) = \frac{1}{\sqrt{2 {πσ}^{2}}} exp ( - \frac{{( x - μ )}^{2}}{2 {σ}^{2}} ) & ( Equation 4 )\end{matrix}$$

$$p ( x ) = \{ \begin{matrix}\frac{1}{b - a} ( a \leq x \leq b ) \\ 0 ( x < a , b < x )\end{matrix} \qquad ( Equation 5 )$$

To set the average of the probability distribution to zero, in the normal distribution of Equation 4, it may be set μ=0, and in the uniform distribution of Equation 5, it may be set a=−b (b>0). In the case of the normal distribution, the standard deviation o is an indicator of the magnitude of the fluctuation. In the case of the uniform distribution, the width of the interval b-a is an indicator of the magnitude of the fluctuation. In other words, the larger this parameter, the greater the fluctuation, and conversely, the smaller this parameter, the smaller the fluctuation. The similarity to the original objective function (optimization problem) also changes depending on the magnitude of the fluctuation applied.

The following describes how fluctuations, represented by random variables that follow the probability distribution shown in Equation 4 or Equation 5, as illustrated above, are applied to the parameters of the first objective function with reference to Equations 1 to 3 above. The fluctuation applied in this example embodiment is expressed as the equation of a random variable x following the fluctuation probability distribution p(x).

For example, it is assumed that the probability distribution p(Xᵢⱼ) of the fluctuation is represented by the normal distribution shown in Equation 4 above. In this case, the second objective function obtained by applying the fluctuation Xᵢⱼ to Equation 1 above is represented by Equation 6 below: As shown in Equation 6, the standard deviation o is set, for example, to a constant c times the parameter aᵢⱼ.

$$\begin{matrix}[ Math . 5 ] & \\ {y}_{i} = {\sum }_{j} ( {a}_{ij} + {X}_{ij} ) ( \frac{{x}_{j} - {\overline{x}}_{j}}{{σ}_{j}} ) + {b}_{i} & ( Equation 6 )\end{matrix}$$

$$p ( {X}_{ij} ) = \frac{1}{\sqrt{2 {πσ}^{2}}} exp ( - \frac{{X}_{ij}^{2}}{2 {σ}^{2}} )$$

$$σ = {ca}_{ij}$$

In Equation 6, the indicator of the magnitude of the fluctuation is the standard deviation σ of p(Xᵢⱼ). Increasing the positive constant c makes it easier to increase the magnitude of fluctuations (i.e., Xᵢⱼ tends to increase).

Similarly, the second objective function obtained by applying the fluctuation Xᵢ to Equation 2 above is represented by Equation 7 below. As shown in Equation 7, the indicator of the magnitude of the fluctuation is set, for example, to a constant c times the parameter Lmedᵢ.

$$\begin{matrix}[ Math . 6 ] & \\ {H}_{o} = {\sum }_{i} {( \frac{{y}_{i} - ( {Lmed}_{i} + {X}_{i} )}{{Lmed}_{i} + {X}_{i}} )}^{2} \cdot {W}_{i} & ( Equation 7 )\end{matrix}$$

$$p ( {X}_{i} ) = \frac{1}{\sqrt{2 {πσ}^{2}}} exp ( - \frac{{X}_{i}^{2}}{2 {σ}^{2}} )$$

$$σ = {cLmed}_{i}$$

The second objective function obtained by applying the fluctuations Xᵢⱼ and Xᵢ to Equation 3 above is represented by Equation 8 below. As shown in Equation 8, the indicator of the magnitude of the fluctuation is set, for example, to constant c times the standard deviation of the parameters Qᵢⱼ and Lᵢ, whichever is not zero.

$$\begin{matrix}[ Math . 7 ] & \\ {H}_{o} = {\sum }_{i} {\sum }_{j \geq i} ( {Q}_{ij} + {X}_{ij} ) {x}_{i} {x}_{j} + {\sum }_{i} ( {L}_{i} + {X}_{i} ) {x}_{i} + const . & ( Equation 8 )\end{matrix}$$

$$p ( {X}_{ij} ) = \frac{1}{\sqrt{2 {πσ}^{2}}} exp ( - \frac{{X}_{ij}^{2}}{2 {σ}^{2}} ) , p ( {X}_{i} ) = \frac{1}{\sqrt{2 {πσ}^{2}}} exp ( - \frac{{X}_{i}^{2}}{2 {σ}^{2}} )$$

$$σ = c \sqrt{\frac{1}{N} ( {\sum }_{{Q}_{ij} \neq 0} {( {Q}_{ij} - \overline{Q} )}^{2} + {\sum }_{{L}_{i} \neq 0} {( {L}_{i} - \overline{Q} )}^{2} )}$$

$$N = {\sum }_{{Q}_{ij} \neq 0} 1 + {\sum }_{{L}_{i} \neq 0} 1 , \overline{Q} = \frac{1}{N} ( {\sum }_{{Q}_{ij} \neq 0} {Q}_{ij} + {\sum }_{{L}_{i} \neq 0} {L}_{i} )$$

The above is an example of the fluctuation equation when the probability distribution is normal. The same applies when the probability distribution is uniform. For example, in the case of Equation 1 above, the probability distribution is represented by Equation 9 below.

$$\begin{matrix}[ Math . 8 ] & \\ p ( {X}_{ij} ) = \{ \begin{matrix}\frac{1}{2 σ} ( - σ \leq {X}_{ij} \leq σ ) \\ 0 ( {X}_{ij} < - σ , σ < {X}_{ij} )\end{matrix} & ( Equation 9 )\end{matrix}$$

Thus, the objective function generation unit 32 generates the second objective function by applying stochastic fluctuations to the parameters of the first objective function. Furthermore, the objective function generation unit 32 may output the generated second objective function (i.e., the objective function with fluctuations applied) and accept modification instructions from engineers etc.

For example, it is assumed that a fluctuation is applied to the parameter aᵢⱼ in Equation 1 above. In this case, the objective function generation unit 32 outputs the generated second objective function after applying the fluctuation to the parameter aᵢⱼ. After the engineer determines the Wᵢ in Equation 2 above, the objective function generation unit 32 may accept the input of the determined Wᵢ as a modification instruction and generate the objective function Ho reflecting the input Wᵢ. At this time, instead of the determined Wᵢ, the objective function generation unit 32 may accept the input of the objective function Ho reflecting Wᵢ as a modification instruction.

Similarly, it is assumed that a fluctuation is applied to the parameter Lmedᵢ in Equation 2 above. In this case, the objective function generation unit 32 outputs the generated second objective function after applying the fluctuation to the parameter Lmedᵢ. After the engineer determines the Wᵢ in Equation 2 above, the objective function generation unit 32 may accept the input of the determined Wi as a modification instruction and generate the objective function Ho reflecting the input Wi. As with the above example, the objective function generation unit 32 may accept the input of the objective function Ho reflecting Wi as a modification instruction instead of the determined Wᵢ.

In this way, by outputting the generated second objective function and accepting modifications from engineers, etc., the objective function after applying fluctuations by engineers, etc., can be verified, thus making it possible to generate a more preferable objective function (mathematical programming problem). In the following description, the objective function modified by engineers is also referred to as the second objective function.

The optimization processing unit 33 performs optimization of the model that includes the second objective function generated by the objective function generation unit 32 and the constraint conditions. Specifically, the optimization processing unit 33 transmits the model to be optimized to the optimization processing device 40 to execute the optimization process and accepts the execution results.

Specifically, first, the optimization processing unit 33 generates the model to be optimized based on the second objective function and constraint conditions, depending on the optimization processing device 40. For example, as described above, it is assumed the optimization processing device 40 is realized by a computer that executes a mathematical programming solver. In this case, the optimization processing unit 33 generates a mathematical optimization problem that includes the second objective function and constraint conditions as the model to be optimized and executes the generated model on the computer.

Alternatively, for example, it is assumed that the optimization processing device 40 is realized by a device that executes annealing (annealing machine) as described above. In this case, the optimization processing unit 33 may generate the Ising model to be optimized based on the second objective function and constraint conditions. The method of generating the Ising model from the objective function and constraint conditions is widely known, so detailed explanation is omitted here.

The output unit 34 outputs the values of the variables of the second objective function obtained by the optimization as a parameter set. These variable values specifically are information indicating the specific values and settings of each factor (e.g., material type and amount, processing temperature, pressure, time). The output unit 34 may output the parameter set directly to the simulator 50 or in a file format (e.g., CSV (Comma Separated Value) format). Additionally, when the optimization processing device 40 is an annealing machine, the optimization result is obtained as binary variables, so the output unit 34 may output the parameter set after converting the optimization result.

The input unit 31, the objective function generation unit 32, the optimization processing unit 33, and the output unit 34 are realized by a computer processor (e.g., CPU (Central Processing Unit)) operating according to the program (parameter generation program).

For example, the program may be stored in the memory unit (not shown) of the parameter generation device 30, and the processor may read the program and operate according to the program, as the input unit 31, the objective function generation unit 32, the optimization processing unit 33, and the output unit 34. The functions of the parameter generation device 30 may also be provided in a Saas (Software as a Service) format.

The input unit 31, the objective function generation unit 32, the optimization processing unit 33, and the output unit 34 may be realized by dedicated hardware. Furthermore, some or all of the components of each device may be realized by a combination of general-purpose or dedicated circuits (circuitry), processors, etc. These may be composed of a single chip or multiple chips connected via a bus. Some or all of the components of each device may be realized by a combination of the above-mentioned circuits, etc., and a program.

When some or all of the components of the parameter generation device 30 are realized by multiple information processing devices or circuits, the multiple information processing devices or circuits may be arranged centrally or distributed. For example, the information processing devices or circuits may be realized as a client-server system, cloud computing system, etc., where each is connected via a communication network.

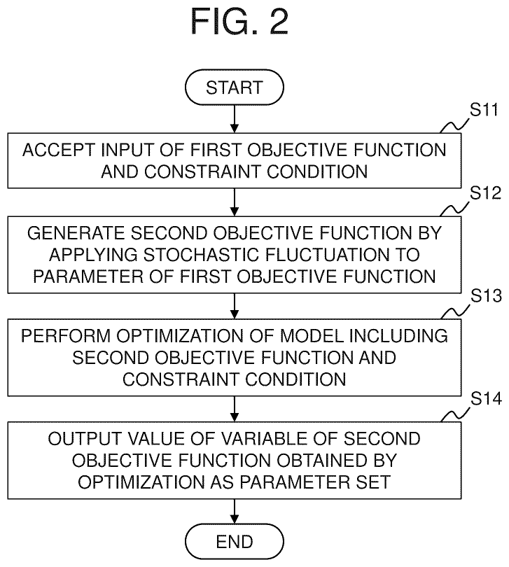

Next, the operation of the parameter generation device 30 according to this example embodiment will be described. FIG. 2 is a flowchart showing an example of the operation of the parameter generation device 30.

The input unit 31 accepts input of the first objective function and constraint conditions (Step S11). As described above, the first objective function is a function that defines combinations of factors related to item production. The objective function generation unit 32 generates the second objective function by applying stochastic fluctuations to the parameters of the first objective function (Step S12). The optimization processing unit 33 performs optimization of the model including the second objective function and constraint conditions (Step S13). Specifically, the optimization processing unit 33 executes optimization processing on the optimization processing device 40. The output unit 34 outputs the values of the variables of the second objective function obtained by the optimization as a parameter set (Step S14).

As described above, in this example embodiment, the input unit 31 accepts input of the first objective function and constraint conditions, and the objective function generation unit 32 generates the second objective function by applying stochastic fluctuations to the parameters of the first objective function. The optimization processing unit 33 performs optimization of the model including the second objective function and constraint conditions, and the output unit 34 outputs the values of the variables of the second objective function obtained by the optimization as a parameter set.

With this configuration, it is possible to obtain a combination of factors (i.e., parameter set) that indicates multiple methods for producing the desired item. By performing simulations based on this parameter set, it is possible to determine whether the desired item is obtained. As a result, it becomes possible to discover multiple methods for producing the desired item.

In this example embodiment, the optimization processing unit 33 causes a computer that performs a mathematical programming solver to perform the optimization processing, thus enabling a fast determination of the parameter set that achieves the desired properties.

Furthermore, in this example embodiment, the objective function generation unit 32 applies fluctuations only for the objective function without changing the constraint conditions, so that a variety of parameter sets that satisfy the constraint conditions can be obtained even in a mathematical programming solver. In this case, the objective function generation unit 32 applies the fluctuations to be applied in the objective function using a probability distribution that is centered on the original model (objective function), so that a parameter set close to the optimal solution for the original model can be obtained.

Moreover, in this example embodiment, by applying fluctuations based on a probability distribution, the objective function generation unit 32 can continuously change the degree of fluctuation, making it possible to obtain diverse parameter sets ranging from those close to the optimal solution to those relatively far from the optimal solution.

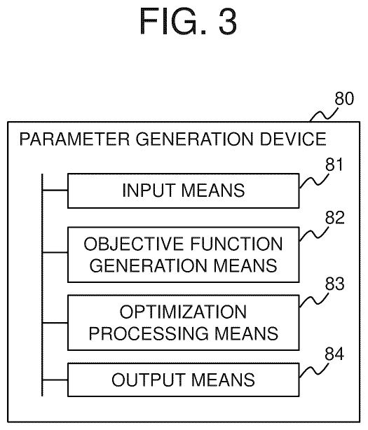

Next, the outline of the invention will be described. FIG. 3 is a block diagram showing an outline of the parameter generation device according to the present invention. The parameter generation device 80 (e.g., the parameter generation device 30) according to the present invention includes an input means 81 (e.g., the input unit 31) which accepts input of a first objective function and a constraint condition (e.g., material type selection, material quantity distribution, exclusive material designation, material processing method, etc.) defining a combination of factors related to item production (e.g., type and amount of material, processing temperature, pressure, time, etc.), an objective function generation means 82 (e.g., the objective function generation unit 32) which generates a second objective function by applying stochastic fluctuation to a parameter of the first objective function, an optimization processing means 83 (e.g., the optimization processing unit 33) which performs optimization of a model including the second objective function and the constraint condition, and an output means 84 (e.g., output unit 34) which outputs a value of a variable of the second objective function obtained by the optimization as a parameter set.

With such a configuration, it becomes possible to discover multiple methods for producing the desired item. In other words, with the above configuration, it is possible to obtain a combination of factors (parameter set) that indicates multiple methods for producing the desired item, and, by performing simulations based on this parameter set, it is possible to determine whether the desired item is obtained. As a result, it becomes possible to discover multiple methods for producing the desired item.

Furthermore, the objective function generation means 82 may generate the second objective function by applying fluctuation represented by a random variable following a predetermined probability distribution to a parameter.

Specifically, the objective function generation means 82 may generate the second objective function by applying fluctuation represented by a random variable following a probability distribution with mean zero to a parameter. With such a configuration, it becomes possible to obtain parameter sets close to the optimal solution for the original model.

Furthermore, the objective function generation means 82 may generate the second objective function by applying fluctuation represented by a random variable following a normal distribution or uniform distribution to a parameter.

Specifically; the objective function generation means 82 may generate the second objective function by applying fluctuation represented by a random variable following a normal distribution where a standard deviation is a constant multiple of a parameter applying fluctuation.

Furthermore, the optimization processing means 83 may generate a mathematical optimization problem as the model to be optimized, including the second objective function and the constraint condition, and causes a computer (e.g., the optimization processing device 40) that performs a mathematical programming solver to perform the generated model. With such a configuration, it becomes possible to rapidly determine a parameter set that achieves the desired properties.

On the other hand, the optimization processing means 83 may generate an Ising model to be optimized based on the second objective function and the constraint condition, and causes an annealing machine (e.g., optimization processing device 40) to execute the generated Ising model. With such a configuration, it becomes possible to obtain parameter sets with different properties from similar objective functions.

Furthermore, the objective function generation means 82 may output the generated second objective function and accept a modification instruction from a user for the second objective function. The optimization processing means 83 may optimize a model to be optimized, including the second objective function and the constraint condition with the modification instruction reflected. With such a configuration, the objective function after applying fluctuations by engineers and others can be verified, making it possible to generate a more preferable objective function (mathematical programming problem).

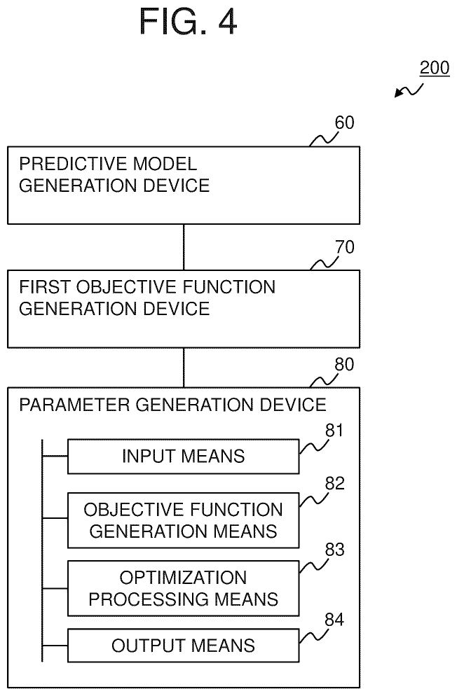

FIG. 4 is a block diagram showing an outline of the simulation system according to the present invention. The simulation system 200 (e.g., the simulation system 100) according to the present invention includes a predictive model generation device 60 (e.g., the predictive model generation device 10) which uses past experimental data as training data to learn a predictive model with material as explanatory variable and characteristic value indicating a property of an item as objective variable, a first objective function generation device 70 (e.g., the first objective function generation device 20) which generates a first objective function defining a combination of factors related to item production using the predictive model, and a parameter generation device 80 (e.g., parameter generation device 30) which generates a parameter set using the first objective function.

The first objective function generation device 70 generates the first objective function, including a linear sum of the characteristic value indicated by the objective variable as the combination of factors, and inputs it into the parameter generation device 80.

The configuration of the parameter generation device 80 is the same as the parameter generation device 80 shown in FIG. 3.

Even with such a configuration, it becomes possible to discover multiple methods for producing the desired item.

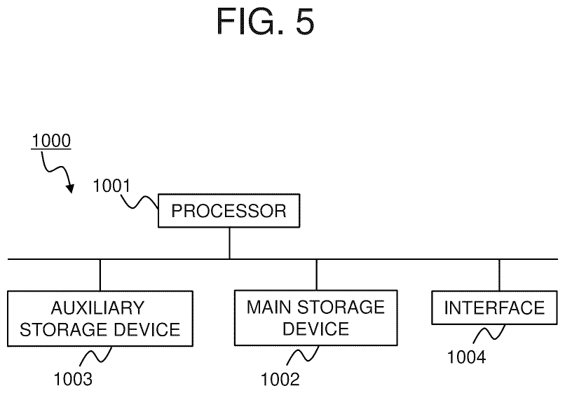

FIG. 5 is a schematic block diagram showing the configuration of a computer according to at least one example embodiment. The computer 1000 includes a processor 1001, a main storage device 1002, an auxiliary storage device 1003, and an interface 1004. Additionally, a computer that executes a mathematical programming solver, an annealing machine, a simulator, etc., may be connected to the computer 1000.

The parameter generation device 80 described above is implemented in the computer 1000. The operations of the above processing units are stored in the auxiliary storage device 1003 in the form of a program (parameter generation program). The processor 1001 reads the program from the auxiliary storage device 1003, loads it into the main storage device 1002, and executes the program according to the above processes.

At least one example embodiment, the auxiliary storage device 1003 is an example of a non-transitory tangible medium. Other examples of non-transitory tangible media include magnetic disks, magneto-optical disks, CD-ROMs (Compact Disc Read-only memory), DVD-ROMs (Read-only memory), semiconductor memories, etc., connected via the interface 1004. When this program is delivered to the computer 1000 via a communication line, the computer 1000 may receive the program, load it into the main storage device 1002, and execute the above processes.

The program may also be for realizing some of the functions described above. Additionally, the program may be a so-called differential file (differential program) that realizes the above-mentioned functions in combination with another program already stored in the auxiliary storage device 1003.

A part of or all of the above example embodiments may also be described as, but not limited to, the following supplementary notes.

(Supplementary note 1) A parameter generation device, including:
an input means which accepts input of a first objective function and a constraint condition defining a combination of factors related to item production,
an objective function generation means which generates a second objective function by applying stochastic fluctuation to a parameter of the first objective function,
an optimization processing means which performs optimization of a model including the second objective function and the constraint condition, and
an output means which outputs a value of a variable of the second objective function obtained by the optimization as a parameter set.

(Supplementary note 2) The parameter generation device according to Supplementary note 1, wherein
the objective function generation means generates the second objective function by applying fluctuation represented by a random variable following a predetermined probability distribution to a parameter.

(Supplementary note 3) The parameter generation device according to Supplementary note 1 or 2, wherein
the objective function generation means generates the second objective function by applying fluctuation represented by a random variable following a probability distribution with mean zero to a parameter.

(Supplementary note 4) The parameter generation device according to any one of Supplementary notes 1 to 3, wherein
the objective function generation means generates the second objective function by applying fluctuation represented by a random variable following a normal distribution or uniform distribution to a parameter.

(Supplementary note 5) The parameter generation device according to any one of Supplementary notes 1 to 4, wherein
the objective function generation means generates the second objective function by applying fluctuation represented by a random variable following a normal distribution where a standard deviation is a constant multiple of a parameter applying fluctuation.

(Supplementary note 6) The parameter generation device according to any one of Supplementary notes 1 to 5, wherein
the optimization processing means generates a mathematical optimization problem as the model to be optimized, including the second objective function and the constraint condition, and causes a computer that performs a mathematical programming solver to perform the generated model.

(Supplementary note 7) The parameter generation device according to any one of Supplementary notes 1 to 5, wherein
the optimization processing means generates an Ising model to be optimized based on the second objective function and the constraint condition, and causes an annealing machine to execute the generated Ising model.

(Supplementary note 8) The parameter generation device according to any one of Supplementary notes 1 to 7, wherein
the objective function generation means outputs the generated second objective function and accepts a modification instruction from a user for the second objective function, and
the optimization processing means optimizes a model to be optimized, including the second objective function and the constraint condition with the modification instruction reflected.

(Supplementary note 9) A parameter generation system includes:
a predictive model generation device which uses past experimental data as training data to learn a predictive model with material as explanatory variable and characteristic value indicating a property of an item as objective variable,
a first objective function generation device which generates a first objective function defining a combination of factors related to item production using the predictive model, and
a parameter generation device which generates a parameter set using the first objective function, wherein
the first objective function generation device generates the first objective function, including a linear sum of the characteristic value indicated by the objective variable as the combination of factors, and
the parameter generation device includes:
an input means which accepts input of the first objective function and a constraint condition,
an objective function generation means which generates a second objective function by applying stochastic fluctuation to a parameter of the first objective function,
an optimization processing means which performs optimization of a model including the second objective function and the constraint condition, and
an output means which outputs a value of a variable of the second objective function obtained by the optimization as a parameter set.

(Supplementary note 10) A parameter generation method by a computer comprising:
accepting input of a first objective function and a constraint condition defining a combination of factors related to item production,
generating a second objective function by applying stochastic fluctuation to a parameter of the first objective function,
performing optimization of a model including the second objective function and the constraint condition, and
outputting a value of a variable of the second objective function obtained by the optimization as a parameter set.

(Supplementary note 11) A program storage medium storing a parameter generation program for causing a computer to execute:
an input process for accepting input of a first objective function and a constraint condition defining a combination of factors related to item production,
an objective function generation process for generating a second objective function by applying stochastic fluctuation to a parameter of the first objective function,
an optimization processing process for performing optimization of a model including the second objective function and the constraint condition, and
an output process for outputting a value of a variable of the second objective function obtained by the optimization as a parameter set.

(Supplementary note 12) A parameter generation program for causing a computer to execute:
an input process for accepting input of a first objective function and a constraint condition defining a combination of factors related to item production,
an objective function generation process for generating a second objective function by applying stochastic fluctuation to a parameter of the first objective function,
an optimization processing process for performing optimization of a model including the second objective function and the constraint condition, and
an output process for outputting a value of a variable of the second objective function obtained by the optimization as a parameter set.

### INDUSTRIAL APPLICABILITY

The present invention is preferably applicable to a parameter generation device that generates desired parameters. Specifically, the present invention is preferably applicable in fields where repeated prototyping or simulation is performed in research site for new item exploration.

### REFERENCE SIGNS LIST

10 Predictive model generation device
11 Storage unit
12 Learning unit
13 Model output unit
20 First objective function generation device
30 Parameter generation device
31 Input unit
32 Objective function generation unit
33 Optimization processing unit
34 Output unit
40 Optimization processing device
50 Simulator

What is claimed is:

1. A parameter generation device, comprising:
a memory storing instructions; and
one or more processors configured to execute the instructions to:
accept input of a first objective function and a constraint condition defining a combination of factors related to item production;
generate a second objective function by applying stochastic fluctuation to a parameter of the first objective function;
perform optimization of a model including the second objective function and the constraint condition; and
output a value of a variable of the second objective function obtained by the optimization as a parameter set.

2. The parameter generation device according to claim 1, wherein the processor is configured to execute the instructions to
generate the second objective function by applying fluctuation represented by a random variable following a predetermined probability distribution to a parameter.

3. The parameter generation device according to claim 1, wherein the processor is configured to execute the instructions to
generate the second objective function by applying fluctuation represented by a random variable following a probability distribution with mean zero to a parameter.

4. The parameter generation device according to claim 1, wherein the processor is configured to execute the instructions to
generate the second objective function by applying fluctuation represented by a random variable following a normal distribution or uniform distribution to a parameter.

5. The parameter generation device according to claim 1, wherein the processor is configured to execute the instructions to
generate the second objective function by applying fluctuation represented by a random variable following a normal distribution where a standard deviation is a constant multiple of a parameter applying fluctuation.

6. The parameter generation device according to claim 1, wherein the processor is configured to execute the instructions to
generate a mathematical optimization problem as the model to be optimized, including the second objective function and the constraint condition, and cause a computer that performs a mathematical programming solver to perform the generated model.

7. The parameter generation device according to claim 1, wherein the processor is configured to execute the instructions to
generate an Ising model to be optimized based on the second objective function and the constraint condition, and cause an annealing machine to execute the generated Ising model.

8. The parameter generation device according to claim 1, wherein the processor is configured to execute the instructions to:
output the generated second objective function and accept a modification instruction from a user for the second objective function; and
optimize a model to be optimized, including the second objective function and the constraint condition with the modification instruction reflected.

9. A parameter generation system comprising:
a predictive model generation device which uses past experimental data as training data to learn a predictive model with material as explanatory variable and characteristic value indicating a property of an item as objective variable;
a first objective function generation device which generates a first objective function defining a combination of factors related to item production using the predictive model; and
a parameter generation device which generates a parameter set using the first objective function, wherein
the first objective function generation device generates the first objective function, including a linear sum of the characteristic value indicated by the objective variable as the combination of factors, and inputs it into the parameter generation device, and
the parameter generation device includes:
a memory storing instructions; and
one or more processors configured to execute the instructions to:
accept input of the first objective function and a constraint condition;
generate a second objective function by applying stochastic fluctuation to a parameter of the first objective function;
perform optimization of a model including the second objective function and the constraint condition; and
output a value of a variable of the second objective function obtained by the optimization as a parameter set.

10. A parameter generation method by a computer comprising:
accepting input of a first objective function and a constraint condition defining a combination of factors related to item production;
generating a second objective function by applying stochastic fluctuation to a parameter of the first objective function;
performing optimization of a model including the second objective function and the constraint condition; and
outputting a value of a variable of the second objective function obtained by the optimization as a parameter set.

11. (canceled)
